(19) United States
(12) Patent Application Publication
(10) Pub. No.: US 2025/0116447 A1
(43) Pub. Date: Apr. 10, 2025

(54) REFRIGERATOR AND REFRIGERATOR AIR DUCT PURIFICATION SYSTEM

(71) Applicant: AUCMA CO., LTD, Qingdao City (CN)

(72) Inventors: Huanjie TENG, Qingdao City (CN); Hongjun SUI, Qingdao City (CN); Ming LIU, Qingdao City (CN); Liran WANG, Qingdao City (CN); Ran CHENG, Qingdao City (CN); Ruitang YE, Qingdao City (CN); Yannan XIE, Qingdao City (CN); Yang DU, Qingdao City (CN)

(21) Appl. No.: 18/695,662

(22) PCT Filed: Dec. 15, 2022
(86) PCT No.: PCT/CN2022/139160

§ 371 Date: Sep. 10, 2024

(30) Foreign Application Priority Data
Jan. 21, 2022 (CN) 202210098482.3

(51) Int. Cl. F25D 17/04; A61L 9/015; F25D 29/00

(52) U.S. Cl. CPC F25D 17/042; A61L 9/015; F25D 29/005; A61L 2209/111; F25D 2317/0416; F25D 2317/067; F25D 2400/36

(57) ABSTRACT

The present disclosure provides a refrigerator and a refrigerator air duct purification system and belongs to the field of refrigerator purification technologies. A refrigeration air duct front foam is fixed at a rear side of a refrigeration air duct backplate. A light-source-oriented light transmission assembly is disposed at a front side of the refrigeration air duct backplate. An air outlet penetrating back and forth is disposed on the refrigeration air duct front foam, the refrigeration air duct backplate and the light-source-oriented light transmission assembly. An outlet air end of the air outlet is in communication with the refrigeration chamber. In a main air duct are disposed an ion generator, an air quality sensor, and an ethylene concentration sensor. A control board is initially in a determination state to determine by a door opening/closing sensor whether a refrigeration door is opened, and if the refrigeration door is opened, start up an ion purification mode. The control board controls the ion generator to work for 3 hours. During the working process of the ion generator, the control board continuously detects the state of the refrigeration door. If the refrigerator door is opened again, the working time of the ion generator is recounted. After the ion generator finishes working, the control board reenters the determination state.

### TECHNICAL FIELD

The present disclosure relates to the field of refrigerator purification technologies and in particular to a refrigerator and a refrigerator air duct purification system.

### BACKGROUND

In the prior arts, after an ion generator of refrigerators starts up a purification mode, the ion generator is always in a working state, which greatly affects the service life of the ion generator. Further, since the refrigerators are mostly used for many years after being purchased, even if the ion generator cannot satisfy the requirements of the user for sterilization and purification after expiration of its service life, the user usually does not change it. The ozone generated by the ion generator has the effect of air purification but it is a gas with a strong odor. Therefore, the user experience will be severely affected.

### SUMMARY

### Technical Problem

### Solution to the Technical Problem

### Technical Solution

The present disclosure discloses a refrigerator and a refrigerator air duct purification system to solve the problem of the short service life and continuous operation of the ion generators in refrigerators in the prior arts.

There is provided a refrigerator which includes a refrigerator body, a chamber, a control board, a refrigeration air duct, a refrigeration air duct backplate, a refrigeration air duct front foam, and a refrigeration air duct rear foam. The chamber includes a refrigeration chamber and a preservation chamber. The refrigeration air duct is disposed between the refrigeration air duct front foam and a back side of the refrigerator body. The refrigeration air duct includes a main air duct and a double air duct. The refrigeration air duct rear foam is disposed at the bottom of the back side of the refrigerator body. An air inlet is disposed on the refrigeration air duct rear foam and the air inlet is in communication with the double air duct.

The refrigeration air duct front foam is fixed at a rear side of the refrigeration air duct backplate. A light-source-oriented light transmission assembly is disposed at a front side of the refrigeration air duct backplate. An air outlet penetrating back and forth is disposed on the refrigeration air duct front foam, the refrigeration air duct backplate and the light-source-oriented light transmission assembly. The air outlet includes an inlet air end and an outlet air end. The top of the main air duct is in communication with the inlet air end of the air outlet, and the bottom of the main air duct is in communication with the double air duct. The outlet air end of the air outlet is in communication with the refrigeration chamber. In the main air duct are disposed an ion generator, an air quality sensor, and an ethylene concentration sensor.

Preferably, the double air duct includes a preservation air duct and a low-temperature air duct. The low-temperature air duct is in communication with the main air duct, and the preservation air duct is in communication with the preservation chamber.

Preferably, an air opening cover is disposed on the air outlet. The air opening cover is covered on the inlet air end of the air outlet. An air opening in communication with the main air duct is disposed at the bottom of the air opening cover.

Preferably, the ion generator, the air quality sensor, and the ethylene concentration sensor are disposed at the bottom of the air opening.

Preferably, a smoke amount sensor is disposed on the refrigeration air duct backplate.

Preferably, a refrigeration door and a preservation door are disposed on a front side of the refrigerator body, and a door opening/closing sensor is disposed at a hinging connection of the refrigeration door and the preservation door respectively.

Preferably, a display panel is disposed at the top of the air outlet and located on a front side of the refrigeration air duct backplate. The display panel, the door opening/closing sensor, the ion generator, the air quality sensor, the ethylene concentration sensor and the smoke amount sensor are all electrically connected to the control board.

There is provided a refrigerator air duct purification system using the refrigerator. The system includes the following steps.

At step S1, the control board is initially in a determination state to determine by the door opening/closing sensor whether the refrigeration door is opened, and if the refrigeration door is opened, start up an ion purification mode.

At step S2, when the refrigeration door is closed, the control board controls the smoke amount sensor to detect a smoke amount inside the refrigerator body; if a measured value of the smoke amount is greater than a set value, the control board determines that the smoke amount is disqualified and starts up the ion purification mode.

At step S3, when the refrigerator door is closed and the smoke amount inside the refrigerator body is qualified, the control board measures a methanthiol content inside the refrigerator body by using the air quality sensor; if a measured value of the methanthiol content is greater than a set value, the control board determines the methanthiol content is disqualified, and starts up the ion purification mode.

At step S4, when the refrigerator door is closed and the smoke amount and the methanthiol content are both qualified, the control board measures an ethylene concentration inside the refrigerator body by using the ethylene concentration sensor; if a measured value of the ethylene concentration is greater than a set value, the control board determines the ethylene concentration is disqualified, and starts up the ion purification mode.

At step S5, when the refrigerator door is closed and the smoke amount, the methanthiol content and the ethylene concentration are all qualified, if the control board detects that the refrigerator door is closed for more than 72 hours and the time is 24:00, the control board starts up the ion purification mode.

Preferably, the ion purification mode is started up in the following specific process.

The control board controls the ion generator to work for 3 hours. During the working process of the ion generator, the control board continuously detects the state of the refrigeration door. If the refrigerator door is opened again, the working time of the ion generator is recounted. After the ion generator finishes working, the control board reenters the determination state.

Preferably, the control board displays the smoke amount, the methanthiol content, the ethylene concentration and the process of the ion purification mode on the display panel.

### Beneficial Effects of the Present Disclosure

### Beneficial Effects

Compared with the prior arts, the present disclosure greatly extends the service life of the ion generator and reduces the product energy consumption. Preferential ion purification at night eliminates the influence of the odor of the ozone generated by the ion generator on the use experience of the users. The purification data can be fed in real time back to the user and the user can use the data to determine whether the ion generator is damaged or reaches its life extreme.

### BRIEF DESCRIPTIONS OF THE DRAWINGS

### Descriptions of the Drawings

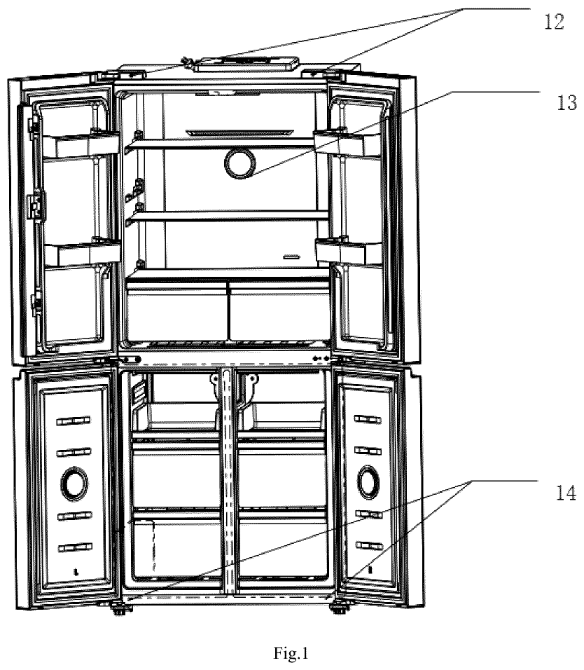

FIG. 1 is a front view of a refrigerator body of the present disclosure.

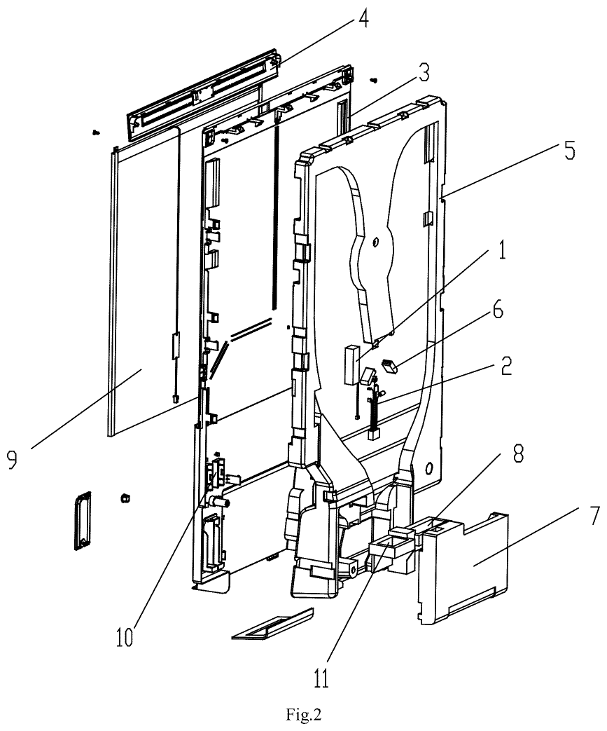

FIG. 2 is an exploded view of relevant components of a refrigeration air duct.

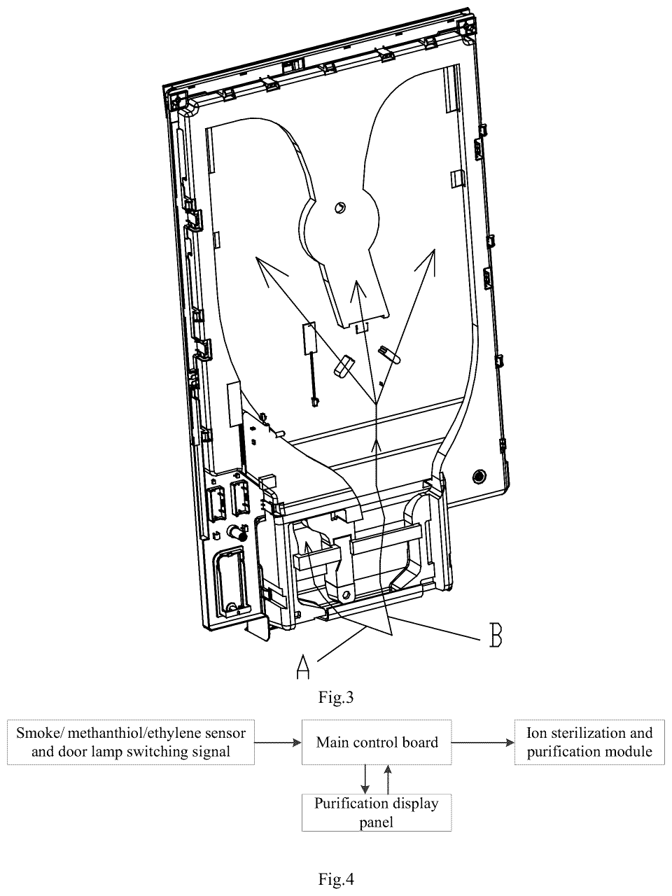

FIG. 3 is a schematic diagram of an air direction of the air duct in FIG. 2.

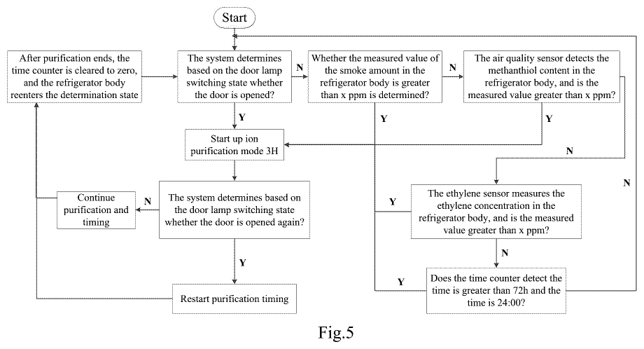

FIG. 4 is a diagram illustrating a control relationship of a control board.

FIG. 5 is a control flowchart of the control board.

Numerals of the drawings are described below: 1. ion generator, 2. air quality sensor, 3. refrigeration air duct backplate, 4. display panel, 5. refrigeration air duct front foam, 6. ethylene concentration sensor, 7. refrigeration air duct rear foam, 8. low-temperature air duct, 9. light-source-oriented light transmission assembly, 10. smoke amount sensor, 11. preservation air duct, 12. door opening/closing sensor of a refrigerator door, 13. air outlet, 14. door opening/closing sensor of a preservation door, A. air direction of the preservation air duct, and B. air direction of the low-temperature air duct and the main air duct.

### EMBODIMENTS OF THE PRESENT DISCLOSURE

### Implementations of the Present Disclosure

The present disclosure will be further detailed below in combination with specific embodiments.

There is provided a refrigerator, which includes a refrigerator body, a chamber, a control board, a refrigeration air duct, a refrigeration air duct backplate 3, a refrigeration air duct front foam 5, and a refrigeration air duct rear foam 7. The chamber includes a refrigeration chamber and a preservation chamber. The refrigeration air duct is disposed between the refrigeration air duct front foam 5 and a back side of the refrigerator body. The refrigeration air duct includes a main air duct and a double air duct. The refrigeration air duct rear foam 7 is disposed at the bottom of the back side of the refrigerator body. An air inlet is disposed on the refrigeration air duct rear foam 7 and the air inlet is in communication with the double air duct.

The refrigeration air duct front foam 5 is fixed at a rear side of the refrigeration air duct backplate 3. A light-source-oriented light transmission assembly 9 is disposed at a front side of the refrigeration air duct backplate 3. An air outlet 13 penetrating back and forth is disposed on the refrigeration air duct front foam 5, the refrigeration air duct backplate 3 and the light-source-oriented light transmission assembly 9. The air outlet 13 includes an inlet air end and an outlet air end. The top of the main air duct is in communication with the inlet air end of the air outlet 13, and the bottom of the main air duct is in communication with the double air duct. The outlet air end of the air outlet 13 is in communication with the refrigeration chamber. In the main air duct are disposed an ion generator 1, an air quality sensor 2, and an ethylene concentration sensor 6.

The double air duct includes a preservation air duct 11 and a low-temperature air duct 8. The low-temperature air duct 8 is in communication with the main air duct, and the preservation air duct 11 is in communication with the preservation chamber.

An air opening cover is disposed on the air outlet 13. The air opening cover is covered on the inlet air end of the air outlet 13. An air opening in communication with the main air duct is disposed at the bottom of the air opening cover.

The ion generator 1, the air quality sensor 2, and the ethylene concentration sensor 6 are disposed at the bottom of the air opening.

A smoke amount sensor 10 is disposed on the refrigeration air duct backplate 3.

A refrigeration door and a preservation door are disposed on a front side of the refrigerator body, and a door opening/closing sensor is disposed at a hinging connection of the refrigeration door and the preservation door respectively.

A display panel 4 is disposed at the top of the air outlet 13 and located on a front side of the refrigeration air duct backplate 3. The display panel 4, the door opening/closing sensor, the ion generator 1, the air quality sensor 2, the ethylene concentration sensor 6 and the smoke amount sensor 10 are all electrically connected to the control board.

There is provided a refrigerator air duct purification system using the refrigerator. The system includes the following steps:

At step S1, the control board is initially in a determination state to determine by the door opening/closing sensor whether the refrigeration door is opened, and if the refrigeration door is opened, start up an ion purification mode.

At step S2, when the refrigeration door is closed, the control board controls the smoke amount sensor 10 to detect a smoke amount inside the refrigerator body; if a measured value of the smoke amount is greater than a set value, the control board determines that the smoke amount is disqualified and starts up the ion purification mode.

At step S3, when the refrigerator door is closed and the smoke amount inside the refrigerator body is qualified, the control board measures a methanthiol content inside the refrigerator body by using the air quality sensor 2; if a measured value of the methanthiol content is greater than a set value, the control board determines the methanthiol content is disqualified, and starts up the ion purification mode.

At step S4, when the refrigerator door is closed and the smoke amount and the methanthiol content are both qualified, the control board measures an ethylene concentration inside the refrigerator body by using the ethylene concentration sensor; if a measured value of the ethylene concentration is greater than a set value, the control board determines the ethylene concentration is disqualified, and starts up the ion purification mode.

At step S5, when the refrigerator door is closed and the smoke amount, the methanthiol content and the ethylene concentration are all qualified, if the control board detects that the refrigerator door is closed for more than 72 hours and the time is 24:00, the control board starts up the ion purification mode.

The ion purification mode is started up in the following specific process.

The control board controls the ion generator 1 to work for 3 hours. During the working process of the ion generator 1, the control board continuously detects the state of the refrigeration door. If the refrigerator door is opened again, the working time of the ion generator 1 is recounted. After the ion generator 1 finishes working, the control board reenters the determination state.

The control board displays the smoke amount, the methanthiol content, the ethylene concentration and the process of the ion purification mode on the display panel 4.

In order to help understand the present disclosure, an internal air direction is described below. After external air enters the air inlet, a part of the external air enters the preservation air duct 11 and the other part of external air enters the low-temperature air duct 8; where the preservation chamber includes a moisture preservation drawer and a freshness preservation drawer, and the air volume of the preservation air duct 11 is used to cool down the moisture preservation drawer and the freshness preservation drawer. The air in the low-temperature air duct 8 enters the main air duct and the air in the main air duct enters the air outlet 13 through the air opening. Two ethylene concentration sensors 6 are disposed at the bottom of the air opening, where the two ethylene concentration sensors 6 are respectively disposed at both sides of the air opening, with their bottoms tilted toward both sides, so as to form an air-through opening with wide bottom and narrow top, helping the air in the main air duct to enter and improving the detection effect of the sensors at the same time. The air quality sensor 2 is disposed at the bottom of the air-through opening of the ethylene concentration sensors 6, and the smoke amount sensor 10 is disposed on the refrigeration air duct backplate 3 to help detect the diffusion of the smoke. The light-source-oriented light transmission assembly 9 is used to illuminate the interior of the refrigeration chamber and the display panel 4 is disposed on the light-source-oriented light transmission assembly 9 to help observe the information on display panel 4.

Of course, the above descriptions are not used to limit the present disclosure and the present disclosure is also not limited to the above examples. All changes, modifications, additions or replacements made by those skilled in the arts within the essence scope of the present disclosure shall fall into the scope of protection of the present disclosure.

1. A refrigerator, comprising a refrigerator body, a chamber, a control board, a refrigeration air duct, a refrigeration air duct backplate, a refrigeration air duct front foam, and a refrigeration air duct rear foam, wherein the chamber comprises a refrigeration chamber and a preservation chamber, the refrigeration air duct is disposed between the refrigeration air duct front foam and a back side of the refrigerator body, the refrigeration air duct comprises a main air duct and a double air duct, the refrigeration air duct rear foam is disposed at the bottom of the back side of the refrigerator body, an air inlet is disposed on the refrigeration air duct rear foam, and the air inlet is in communication with the double air duct;
the refrigeration air duct front foam is fixed at a rear side of the refrigeration air duct backplate, a light-source-oriented light transmission assembly is disposed at a front side of the refrigeration air duct backplate, an air outlet penetrating back and forth is disposed on the refrigeration air duct front foam, the refrigeration air duct backplate and the light-source-oriented light transmission assembly, the air outlet comprises an inlet air end and an outlet air end, the top of the main air duct is in communication with the inlet air end of the air outlet, the bottom of the main air duct is in communication with the double air duct, the outlet air end of the air outlet is in communication with the refrigeration chamber, and in the main air duct are disposed an ion generator, an air quality sensor, and an ethylene concentration sensor.

2. The refrigerator of claim 1, wherein the double air duct comprises a preservation air duct and a low-temperature air duct; the low-temperature air duct is in communication with the main air duct, and the preservation air duct is in communication with the preservation chamber.

3. The refrigerator of claim 2, wherein an air opening cover is disposed on the air outlet; the air opening cover is covered on the inlet air end of the air outlet; and an air opening in communication with the main air duct is disposed at the bottom of the air opening cover.

4. The refrigerator of claim 3, wherein the ion generator, the air quality sensor, and the ethylene concentration sensor are disposed at the bottom of the air opening.

5. The refrigerator of claim 4, wherein a smoke amount sensor is disposed on the refrigeration air duct backplate.

6. The refrigerator of claim 5, wherein a refrigeration door and a preservation door are disposed on a front side of the refrigerator body, and a door opening/closing sensor is disposed at a hinging connection of the refrigeration door and the preservation door respectively.

7. The refrigerator of claim 6, wherein a display panel is disposed at the top of the air outlet and located on a front side of the refrigeration air duct backplate, and the display panel, the door opening/closing sensor, the ion generator, the air quality sensor, the ethylene concentration sensor and the smoke amount sensor are all electrically connected to the control board.

8. A refrigerator air duct purification system, using the refrigerator of any one of claims 1 to 7, and comprising the following steps:
the control board is initially in a determination state to determine by the door opening/closing sensor whether the refrigeration door is opened, and if the refrigeration door is opened, start up an ion purification mode;
when the refrigeration door is closed, the control board controls the smoke amount sensor to detect a smoke amount inside the refrigerator body; if a measured value of the smoke amount is greater than a set value, the control board determines that the smoke amount is disqualified and starts up the ion purification mode;
when the refrigerator door is closed and the smoke amount inside the refrigerator body is qualified, the control board measures a methanthiol content inside the refrigerator body by using the air quality sensor; if a measured value of the methanthiol content is greater than a set value, the control board determines the methanthiol content is disqualified, and starts up the ion purification mode;
when the refrigerator door is closed and the smoke amount and the methanthiol content are both qualified, the control board measures an ethylene concentration inside the refrigerator body by using the ethylene concentration sensor; if a measured value of the ethylene concentration is greater than a set value, the control board determines the ethylene concentration is disqualified, and starts up the ion purification mode;
when the refrigerator door is closed and the smoke amount, the methanthiol content and the ethylene concentration are all qualified, if the control board detects that the refrigerator door is closed for more than 72 hours and the time is 24:00, the control board starts up the ion purification mode.

9. The refrigerator air duct purification system of claim 8, wherein the ion purification mode is started up in the following specific process:
the control board controls the ion generator to work for 3 hours; during the working process of the ion generator, the control board continuously detects the state of the refrigeration door; if the refrigerator door is opened again, the working time of the ion generator is recounted; after the ion generator finishes working, the control board reenters the determination state.

10. The refrigerator air duct purification system of claim 9, wherein the control board displays the smoke amount, the methanthiol content, the ethylene concentration and the process of the ion purification mode on the display panel.
